(19) United States
(12) Patent Application Publication
(10) Pub. No.: US 2022/0228788 A1
(43) Pub. Date: Jul. 21, 2022

(54) FREEZING DEVICE AND A METHOD USING THE SAME

(71) Applicant: CRYORTH CO., LTD., Taipei City (TW)

(72) Inventors: Yu-Chi CHENG, Taipei City (TW); Shu-Mei HSU, Taipei City (TW); Po-Kuei WU, Taipei City (TW); Kuang-Yu CHAO, Taipei City (TW)

(21) Appl. No.: 17/579,448

(22) Filed: Jan. 19, 2022

(30) Foreign Application Priority Data
Jan. 21, 2021 (TW) 110102369

(51) Int. Cl. F25D 3/10; F25C 1/22; F25C 1/04

(52) U.S. Cl. CPC F25D 3/10; F25C 2301/00; F25C 1/04; F25C 1/22

(57) ABSTRACT

The disclosure provides a freezing device for freezing a liquid mixture and a method using the same. The freezing device includes a container and a tray removably disposed in the container. The tray has a tray plate disposed above a container bottom wall and has a plurality of spaced-apart receptacles for receiving the liquid mixture. When liquid nitrogen is introduced into a region between the tray plate and a container bottom wall, the liquid mixture received in the receptacles is solidified by the liquid nitrogen and is formed into shaped solid coolant pieces.

### CROSS-REFERENCE TO RELATED APPLICATION

This application claims priority to Taiwanese Patent Application No. 110102369, filed on Jan. 21, 2021.

### FIELD

The disclosure relates to a freezing device, and more particularly to a freezing device for freezing a liquid to produce shaped solid coolant pieces and a method using the same.

### BACKGROUND

In recent years, cryosurgery is commonly used to reduce the chance of a tumor recurrence after surgery. Liquid nitrogen with a temperature lower than −196° C. is used to directly contact and destroy cells surrounding an excision site. However, the rapid atomization and flowability of the liquid nitrogen can easily cause unclear surgical vision that results in large-scale necrosis of normal tissues around the excision site and other complications.

According to clinical research reports, malignant tumor cells can be destroyed at a temperatures below −60° C., and the use of liquid nitrogen as a medical treatment is not absolutely necessary. Instead, solid ethanol at a temperature lower than −114° C. can be used to contact and destroy the cells around the excision site. To solidify the ethanol, an ethanol-water solution is directly poured into and stirred with a low temperature liquid nitrogen until a thermal equilibrium is reached, thereby forming a solidified ethanol mixture. However, because only part of the ethanol-water solution is solidified, this method encounters difficulties in obtaining a completely solidified ethanol-water mixture.

### SUMMARY

One object of the disclosure is to provide a freezing device for freezing a liquid mixture so as to produce a shaped solid coolant piece, such as a solid coolant piece for cryotherapy.

According to the object, a freezing device includes a container and a tray.

The container defines a receiving space which opens upward and has a container bottom wall underlying the receiving space.

The tray is removably disposed in the receiving space and has a tray plate situated above the container bottom wall in a spaced apart manner. The tray plate has an upper panel part, and a plurality of spaced-apart receptacles for receiving the liquid mixture. The upper panel part interconnects the receptacles and isolates interior spaces of the receptacles from a bottom region of the receiving space between the tray plate and the container bottom wall.

When liquid nitrogen is introduced into a region between the tray plate and the container bottom wall, the liquid mixture received in the receptacles is solidified by heat absorption and vaporization of the liquid nitrogen received in the receiving space.

Another object of the disclosure is to provide a method of freezing a liquid mixture to produce a shaped solid coolant piece, such as a shaped solid coolant piece for cryotherapy.

Accordingly, a method of the disclosure includes providing a container defining a receiving space which opens upward, and having a container bottom wall underlying the receiving space; positioning a tray in the receiving space, the tray having a tray plate situated above the container bottom wall in a spaced apart manner, and having a plurality of spaced apart receptacles; filling the receptacles with a liquid mixture; introducing liquid nitrogen into a region between the tray plate and the container bottom wall until the liquid nitrogen contacts the receptacles, thereby solidifying the liquid mixture.

### BRIEF DESCRIPTION OF THE DRAWINGS

Other features and advantages of the disclosure will become apparent in the following detailed description of the embodiment with reference to the accompanying drawings, of which:

FIG. 1 is a perspective view of a freezing device according to an embodiment of the disclosure illustrating a cover to cover a tray connected to a container;

FIG. 2 is an exploded perspective view of the embodiment illustrating the tray and the container of the freezing device;

FIG. 3 is a sectional view of the embodiment illustrating probes disposed in the tray attached to the container of the freezing device; and

FIG. 4 is a perspective view of the embodiment illustrating a variant of the tray of the freezing device.

### DETAILED DESCRIPTION

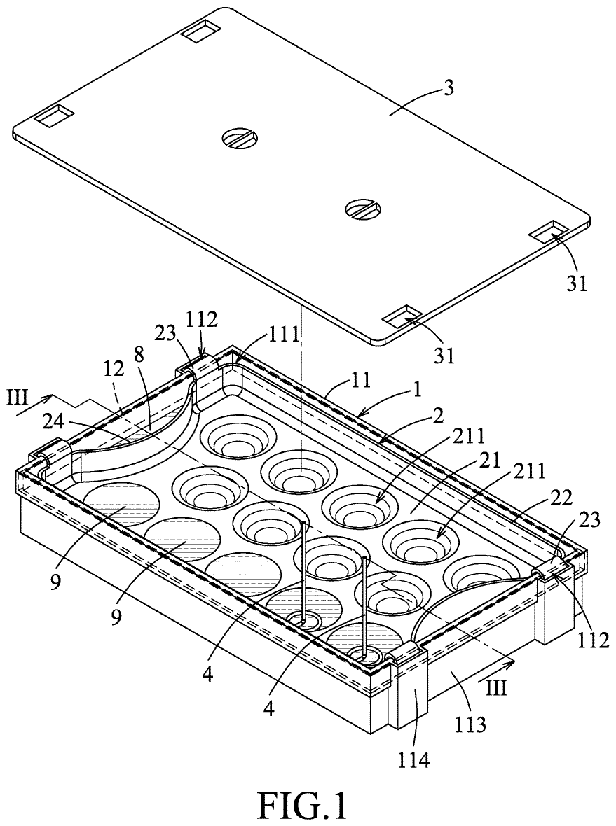

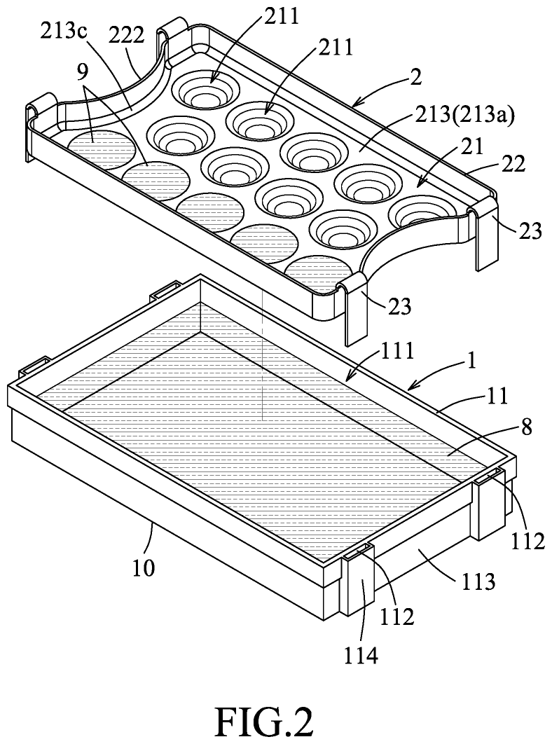

FIGS. 1 to 3 illustrates a freezing device for separately receiving a liquid mixture 9 and liquid nitrogen 8 to solidify the liquid mixture 9 therein. In this embodiment, the liquid mixture 9 after being solidified is used for cryotherapy treatment of tumor. The liquid mixture 9 includes an ethanol water solution and liquid nitrogen; the ethanol water solution has a concentration of more than 80%; a volume ratio of the ethanol water solution to liquid nitrogen ranges from 1:1 to 1:5. By adding liquid nitrogen to the ethanol water solution, a temperature of the liquid mixture 9 can be quickly reduced to a freezing point (e.g. −110° C.). In addition, after being solidified, the liquid mixture 9 can be easily demolded. The liquid mixture 9 may also include, but not limited to, another substance, for example, iodine or isopropyl alcohol.

The freezing device of the disclosure includes a container 1, a tray 2, a top cover 3, and a plurality of probes 4.

The container 1 has a container bottom wall 10, a container surrounding wall 11 extending upwardly from the container bottom wall 10, and a reinforcement member 12. The container 1 defines a receiving space 111 which opens upward to receive the liquid nitrogen 8. The container bottom wall 10 underlies the receiving space 111. The container surrounding wall 11 has an outer surface 113, and a plurality of hollow members 114 formed on the outer surface 113 and respectively defining insertion holes 112. The insertion holes 112 open at a top end of the container surrounding wall 11 and are formed outside of the receiving space 111. In this embodiment, the container 1 is exemplified as having a depth of 4.3 cm in the receiving space 111 and a wall thickness of 2.5 mm. Each insertion hole 112 extend through top and bottom of the corresponding hollow member 114. The container 1 is made of a thermal insulation material, e.g. a silicone rubber, having a poor thermal conductivity, so that a heat exchange rate between the receiving space 111 and an external environment can be reduced to prevent the liquid nitrogen S received in the container 1 from rapidly vaporizing and escaping. Because the container 1 is made of a thermal insulation material, frostbite of a person's skin due to contacting with the container 1 can be avoided. The reinforcement member 12 is embedded in an upper portion of the container surrounding wall 11 of the container 1 to maintain the shape of the container 1. Particularly, the reinforcement member 12 is made of a metal material and loops around the receiving space 111 to avoid deformation of the container surrounding wall 11. Because the reinforcement member 12 is embedded in the container 1, a user can avoid accidentally contacting the reinforcement member 12 which is cooled to a freezing temperature by liquid nitrogen, thereby reducing the risk of skin injury.

The tray 2 is removably disposed in the receiving space 111, and has a tray plate 21 situated above the container bottom wall 10 in a spaced apart manner, a tray surrounding wall 22 extending upwardly from the tray plate 21, and a plurality of hanging hooks 23 connected to the tray surrounding wall 22.

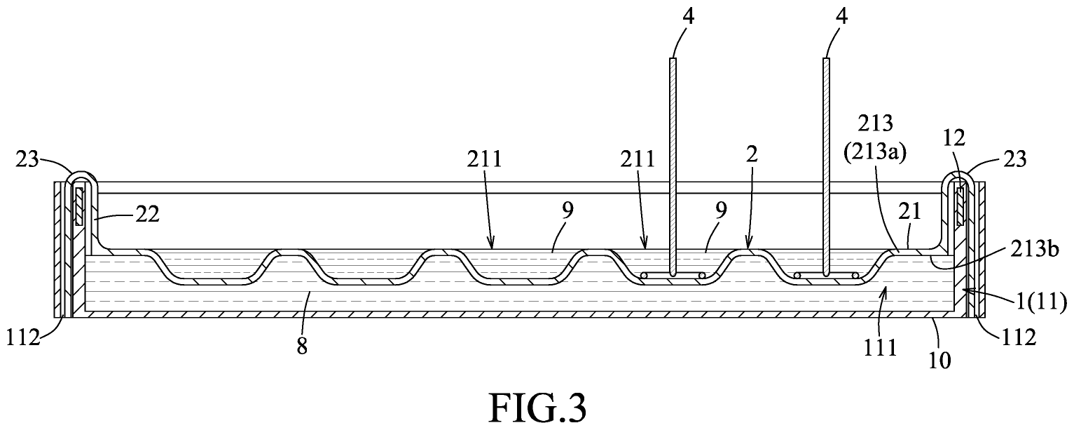

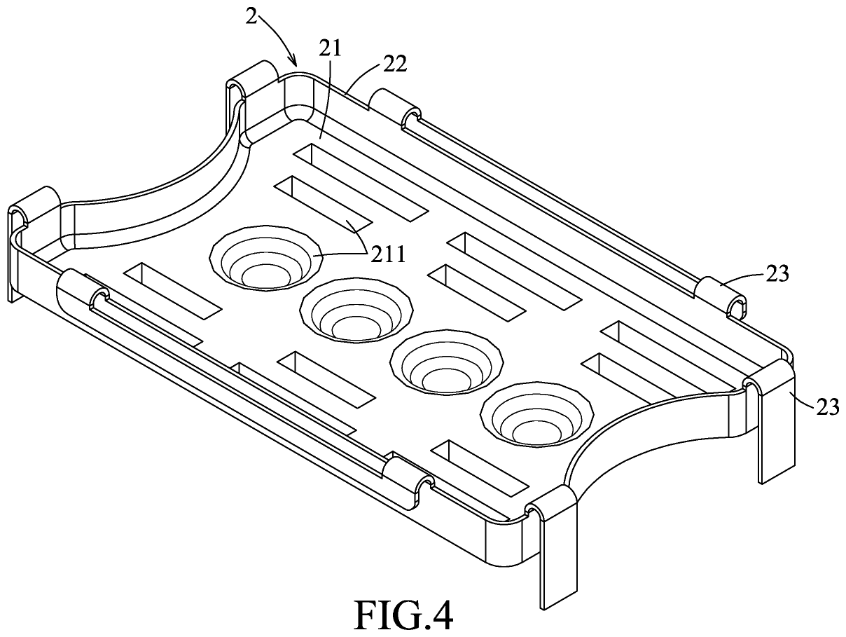

The tray plate 21 has an upper panel part 213, and a plurality of spaced-apart receptacles 211 for receiving the liquid mixture 9. The receptacles 211 are interconnected by the upper panel part 213. As shown in FIG. 3, the upper panel part 213 has a top surface 213a and a bottom surface 213b. The receptacles 211 open at the top surface 213a and protrudes downward from the bottom surface 213b. The upper panel part 213 serves as a partition to isolate interior spaces of the receptacles 211 from a bottom region of the receiving space 111 between the upper panel part 213 of the tray plate 21 and the container bottom wall 10. When liquid nitrogen 8 is introduced into the bottom region between the tray plate 21 and the container bottom wall 10, the liquid mixture 9 received in the receptacles 211 is solidified by heat absorption and vaporization of the liquid nitrogen S received in the bottom region of the receiving space 111. As shown in FIG. 1, each receptacle 211 is in a truncated cone shape, and is exemplified as having an opening with a diameter of 3.8 cm and a volume of 15 ml. FIG. 4 illustrates a variant in which some receptacles 211 are formed into a rectangular parallelepiped shape. However, the shape of each receptacle 211 is not limited hereto, and the number of the receptacles 211 is not limited by this disclosure.

When the tray 2 is disposed in the receiving space 111, the top surface 213a of the upper panel part 213 of the tray plate 21 is lower than the top end of the container surrounding wall 11 so that the bottom surface 213b of the tray plate 21 and the receptacles 211 are immersed in the liquid nitrogen received in the bottom region of the receiving space 111 of the container 1. As such, a contact area between the tray 2 and the liquid nitrogen 8 can be increased to increase a cooling rate of the liquid mixture 9 in the receptacles 211 so that the temperature of the solidified liquid mixture 9 can be maintained and the solidified liquid mixture 9 cannot melt easily. In this embodiment, each receptacle 211 is exemplified as having a depth of 2 cm. When the liquid nitrogen 8 in the receiving space 111 gradually vaporize, the receptacles 211 can still be partially immersed in the liquid nitrogen 8. Further, a wall thickness of each receptacle 211 preferably ranges between 0.5 mm and 0.8 mm. When the wall thickness of each receptacle 211 is 0.8 mm, heat transfer is facilitated but not overly fast. The wall thickness of each receptacle 211 may also be greater than 0.8 mm in order to solidify the liquid mixture 9 at a lower speed to prevent the vaporized liquid nitrogen from being wrapped into the liquid mixtures 9. When the thickness of each receptacle 211 ranges between 0.5 mm and 0.8 mm, the liquid mixture 9 received in the receptacles 211 can be quickly solidified by heat absorption of the liquid nitrogen 8 received in the receiving space 111, but will not produce cracks in the solidified liquid mixture 9.

The tray surrounding wall 22 extends upward from and loops along an outer periphery of the upper panel part 213 of the tray plate 21 to prevent the liquid mixture 9 from overflowing outward from the tray 2 and also prevent the liquid nitrogen 8 from flowing into the receptacles 211. When the tray 2 is disposed in the receiving space 111, the tray surrounding wall 22 is surrounded by the container surrounding wall 11. The tray surrounding wall 22 and the container surrounding wall 11 defines therebetween two passageways 24 for passage of liquid or gaseous nitrogen. Each passageway 24 opens at the top end of the container surrounding wall 11 and communicates the bottom region of the receiving space 111 between the tray plate 21 and the container bottom wall 10. In this embodiment, the upper panel part 213 of the tray plate 21 has two indenting parts 213c respectively formed in two opposite sides of the outer periphery thereof, and the tray surrounding wall 22 has two indenting parts 222 respectively formed in two opposite sides thereof. Each indenting part 213c and each indenting part 222 indents inwardly to extend away from the container surrounding wall 11 so that two passageways 24 are formed between the tray surrounding wall 22 and the container surrounding wall 11. Each passageway 24 allows the vaporized liquid nitrogen 8 in the receiving space 111 to flow outward from the container 1. The liquid nitrogen 8 can be introduced into the receiving space 111 through the passageways 24. In other embodiments, the number of the passageways 24 may be one or more than three.

In this embodiment, each of the hanging hooks 23 protrudes sideward from a top end of the tray surrounding wall 22, passes through the top end of the container surrounding wall 11, and then extends downward to enter a respective one of the insertion holes 112 in a removable manner so that the tray 2 is stably hung on the container surrounding wall 11. The length of each hanging hook 23 inserted into the insertion hole 112 is arranged to correspond to the height of the container surrounding wall 11 so as to support and strengthen the container surrounding wall 11 and prevent deformation of the container surrounding wall 11. However, the insertion holes 112 are not absolutely necessary. In other embodiments, the hanging hooks 23 can be removably and directly attached to the top end of the container 1, and the insertion holes 112 are omitted. As shown in FIG. 1, the tray surrounding wall 22 is four-sided, and the hanging hooks 23 are disposed on two sides of the tray surrounding wall 22 which are opposite along one of a lengthwise direction and a widthwise direction of the tray 2. In some embodiments, as shown in FIG. 4, the hanging hooks 23 are disposed on four sides of the tray surrounding wall 22. When the hanging hooks 23 are attached to the top end of the container surrounding wall 11, the opening of the receiving space 111 is prevented from deforming.

The tray 2, in this embodiment, is made of a stainless steel which can provide good heat conduction for rapidly attaining a thermal equilibrium between the liquid mixture in the receptacles 211 and the liquid nitrogen in the receiving space 111, and for increasing the rate of solidifying the liquid mixture. The tray 2 made of stainless steel has good corrosion and heat resisting properties and good low temperature strength. Therefore, the tray 2 can be sterilized by using high temperature heat, or disinfectants, and will not deform when contacting extremely low temperature liquid nitrogen.

The top cover 3 is removably disposed on the top end of the container 1 to cover the receiving space 111 of the container 1. After the tray 2 is sterilized, it can be placed in the receiving space 111 of the container 1 and is covered by the top cover 3 to avoid exposure to an external environment which can cause contamination. The top cover 3 has a plurality of through holes 31 respectively aligned with the hanging hooks 23 so that top parts of the hanging hooks 23 can protrude outwardly and respectively from the through holes 23, thereby allowing the top cover 3 to tightly abut against the top end of the container 1.

The probes 4 are respectively and removably disposed in the receptacles 211 in an upright manner. In the drawings, only two probes 4 respectively inserted into two of the receptacles 211 are shown as an exemplification. When the liquid mixture 9 in the receptacles 211 is cooled and solidified around the probes 4, the probes 4 are inserted into the solidified liquid mixture 9. The probe 4 is convenient for removal of the solidified pieces of the liquid mixture 9 from the receptacles 211.

In use, the tray 2 is placed in the receiving space 111, and the hanging hooks 23 are respectively inserted into the insertion holes 112 for the tray 2 to be hung in the receiving space 111. The liquid mixture 9, which is premixed and pre-cooled, but not solidified, is filled in the receptacles 211 (see one of three rows of the receptacles 211 in FIG. 1) in a manner that the surface of the liquid mixture 9 is not higher than the upper panel part 213 of the tray plate 21 so that the shape of the liquid mixture after being solidified conforms to that of the receptacle 211. Subsequently, the liquid nitrogen 8 is introduced through the passageways 24 into the receiving space 111 in a manner that the tray plate is immersed in the liquid nitrogen 8 and the liquid level of the liquid nitrogen 8 is not higher than the tray plate 21. By virtue of heat conduction of the tray 2 made of the stainless steel, the liquid mixture 9 in the receptacles 211 can quickly reach a thermal equilibrium with the liquid nitrogen 8 and is cooled to minus 114° C. and solidified. A portion of the liquid nitrogen 8 received in the receiving space 111, which is converted into gaseous nitrogen due to heat absorption, will escape from the container 1 through the passageways 24. After the liquid mixture 9 is solidified, the tray 2 may be removed from the container 1, and the solidified liquid mixture 9 may be removed from the tray 2. As the liquid mixture 9 is solidified in the receptacles 211, it is shaped by the receptacles 211 and formed into multiple shaped solid coolant pieces. Due to the smooth surface of the stainless steel tray 2, the shaped solid coolant pieces can be easily removed from the tray 2.

The freezing device of the disclosure is capable of quickly solidifying and forming the liquid mixture into multiple shaped solid coolant pieces and therefore can be used for preparing the solid coolant pieces in an operation room for cryosurgery. As the solid coolant pieces can be quickly prepared and easily taken out from the receptacles 211, it is not necessary to prepare the solid coolant pieces too early before cryotherapy and premature melting of the solid coolant pieces can be avoided.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A freezing device for freezing a liquid mixture to produce a shaped solid coolant piece, comprising:
a container defining a receiving space which opens upward and having a container bottom wall underlying said receiving space; and
a tray removably disposed in said receiving space and having a tray plate situated above said container bottom wall in a spaced apart manner, said tray plate having an upper panel part, and a plurality of spaced-apart receptacles for receiving the liquid mixture, said upper panel part interconnecting said receptacles and isolating interior spaces of said receptacles from a bottom region of said receiving space between said tray plate and said container bottom wall;
wherein, when liquid nitrogen is introduced into said bottom region between said tray plate and said container bottom wall, the liquid mixture received in said receptacles is solidified by heat absorption and vaporization of the liquid nitrogen received in said bottom region of said receiving space.

2. The freezing device as claimed in claim 1, wherein said tray further has a tray surrounding wall extending upwardly from and looping along an outer periphery of said upper panel part of said tray plate and surrounding said receptacles.

3. The freezing device as claimed in claim 2, wherein:
said tray further has a plurality of hanging hooks connected to said tray surrounding wall; and
said hanging hooks are removably connected to a top end of said container so that said tray is hung in said receiving space.

4. The freezing device as claimed in claim 3, wherein
said container further has a container surrounding wall extending upwardly from said container bottom wall and surrounding said tray surrounding wall,
and a plurality of insertion holes opening at a top end of said container surrounding wall and formed outside of said receiving space; and
said hanging hooks of said tray are respectively and removably inserted into said insertion holes so that said tray is hung on said container surrounding wall.

5. The freezing device as claimed in claim 4, wherein said container surrounding wall has an outer surface, and a plurality of hollow members formed on said outer surface and respectively defining said insertion holes.

6. The freezing device as claimed in claim 2, wherein said container further has a container surrounding wall extending upwardly from said container bottom wall and surrounding said tray surrounding wall, said container surrounding wall and said tray surrounding wall defining therebetween at least one passageway for passage of liquid or gaseous nitrogen, said at least one passageway opening at the top end of said container and communicating with said bottom region of said receiving space between said upper panel part of said tray plate and said container bottom wall.

7. The freezing device as claimed in claim 6, wherein said upper panel part of said tray plate has at least one indenting part in said outer periphery, said tray surrounding wall having at least one indenting part, said indenting parts of said upper panel part and said tray surrounding wall indenting inwardly to extend away from said container surrounding wall so that said at least one passageway is formed between said tray surrounding wall and said container surrounding wall.

8. The freezing device as claimed in claim 1, wherein said container is made of a thermal insulation material.

9. The freezing device as claimed in claim 1, wherein said container is made of a silicone rubber, and said tray is made of a stainless steel.

10. The freezing device as claimed in claim 4, wherein said container further has a reinforcement member embedded in an upper portion of said container surrounding wall of said container to maintain a shape of said container, said reinforcement member looping around said receiving space.

11. The freezing device as claimed in claim 4, wherein said upper panel part of said tray plate is lower than said top end of said container surrounding wall so that said tray can be immersed in the liquid nitrogen received in said bottom region of said receiving space between said upper panel part of said tray plate and said container bottom wall.

12. The freezing device as claimed in claim 1, wherein said upper panel part has a top surface and a bottom surface, said receptacles opening at said top surface and protruding downward from said bottom surface.

13. The freezing device as claimed in claim 4, wherein said hanging hooks are disposed on two sides of said tray surrounding wall which are opposite along one of a lengthwise direction and a widthwise direction of said tray.

14. The freezing device as claimed in 13, wherein each of said hanging hooks protrudes sideward from a top end of said tray surrounding wall, passes through said top end of said container surrounding wall and then extends downward to enter a respective one of said insertion holes.

15. The freezing device as claimed in claim 1, further comprising a top cover removably disposed on a top end of said container to cover said receiving space of said container.

16. The freezing device as claimed in claim 15, wherein said top cover has a plurality of through holes respectively aligned with said hanging hooks.

17. The freezing device as claimed in claim 1, further comprising a plurality of probes respectively and removably disposed in said receptacles in an upright manner.

18. The freezing device as claimed in claim 10, wherein a thickness of each of said receptacles is not smaller than 0.5 mm.

19. The freezing device as claimed in 1, wherein each of said receptacles is configured to receive an ethanol water mixture.

20. A method of freezing a liquid mixture to produce a shaped solid coolant piece, comprising:
providing a container defining a receiving space which opens upward, and having a container bottom wall underlying the receiving space;
positioning a tray in the receiving space, the tray having a tray plate situated above the container bottom wall in a spaced apart manner, and having a plurality of spaced apart receptacles;
filling the receptacles with a liquid mixture; and
introducing liquid nitrogen into a bottom region of the receiving space between the tray plate and the container bottom wall until the liquid nitrogen contacts the receptacles, thereby solidifying the liquid mixture.
